(19) United States
(12) Patent Application Publication
(10) Pub. No.: US 2013/0310687 A1
(43) Pub. Date: Nov. 21, 2013

(54) BLOOD VESSEL EMBOLIZATION METHOD USING BALLOON CATHETER AND BALLOON CATHETER FOR BLOOD VESSEL EMBOLIZATION METHOD

(71) Applicants: Kenji TAKIZAWA, Tokyo (JP); Yukihiko Murata, Ichinomiya-shi (JP)

(72) Inventors: Kenji TAKIZAWA, Tokyo (JP); Yukihiko Murata, Ichinomiya-shi (JP)

(73) Assignees: TERUMO CLINICAL SUPPLY CO., LTD., Kakamigahara-shi (JP); ST. MARIANNA UNIVERSITY SCHOOL OF MEDICINE, Kawasaki-shi (JP)

(21) Appl. No.: 13/476,522

(22) Filed: May 21, 2012

(51) Int. Cl. A61M 25/10; A61B 6/00

(52) U.S. Cl. 600/435; 604/509; 604/97.02

(57) ABSTRACT

A blood vessel embolization method includes inserting a balloon catheter into a blood vessel of a human or an animal having a lesion site and disposing a balloon of the balloon catheter at a portion located at a proximal side of the blood vessel and in a neighborhood of the lesion site thereof; shutting off a blood flow in the blood vessel by expanding the balloon; discharging a glucose solution from a distal end of the balloon catheter in a state in which the blood flow in the blood vessel is shut off; discharging a cyanoacrylate-based embolization substance-containing liquid from the distal end of the balloon catheter after the glucose solution injection step; hardening an embolization substance by maintaining a blood flow shut-off state of the blood vessel after the embolization substance injection step; and removing the balloon catheter from the blood vessel after the embolization substance-hardening step.

### BACKGROUND OF THE INVENTION

1. Field of the Invention

The present invention relates to a blood vessel embolization method of embolizing blood vessels having a lesion site such as a bleeding site, an aneurysm, a blood vessel malformation, and a tumor by using a cyanoacrylate-based embolization substance and a balloon catheter to be used in a blood vessel embolization method.

2. Description of the Related Arts

It is effective in various clinical therapy to form an embolization in a blood vessel. As blood vessels to be embolized, it is possible to list trauma-caused bleeding, bleeding caused by rupture of tumors, bleeding caused by blood vessel diseases (aneurysm rupture, blood vessel malformation), advance occlusion of blood vessel diseases (aneurysm rupture, blood vessel malformation), blood vessels of the abdominal organs having lesion sites (for example, tumor). The artery in the liver, the lineal artery, the renal artery, the internal iliac artery, the arteries of four limbs, and branching blood vessels of these arteries, and the lumbar artery, and the intercostals artery are representative of the blood vessels to which the blood vessel embolization method of the present invention is applicable.

For example, the blood vessel embolization forming method using the removable balloon fed into the aneurysm site by the catheter disposed in a blood vessel is disclosed in U.S. Pat. No. 4,819,637. In this method, the balloon mounted at the distal portion of the catheter is delivered into the aneurysm in which the balloon is expanded by the coagulable fluid (normally polymerizable gel) to embolize the aneurysm. Thereafter by applying a weak traction force to the catheter, the balloon is separated from the catheter. Although the above-described balloon-type embolization forming device is capable of effectively embolizing many kinds of aneurysms, it is difficult to pull out the balloon or move the balloon after the coagulable fluid hardens.

As a blood vessel embolization method, a method directly injecting an embolization forming agent consisting of a liquid polymer into a blood vessel to be embolized is known. As the liquid polymer to be used in the direct injection method, a rapid polymerizable liquid such as cyanoacrylate (superglue consisting of acrylic monomer) and particularly N-butyl cyanoacrylate (NBCA) is used.

The NBCA is an excellent liquid embolization substance and effective as the substance for embolizing a blood vessel malformation having a fast blood flow and aneurysm and varix. The NBCA injected into the blood vessel from a catheter polymerizes instantaneously when it contacts blood and becomes a solid substance, thus occluding the vascular lumen. But it is very difficult to adjust a timing of polymerization. The NBCA occludes at the proximal side of the blood or flows to the vein. There is a case in which the NBCA occludes the catheter.

### SUMMARY OF THE INVENTION

Therefore it is an object of the present invention to provide a blood vessel embolization method which is to be carried out by using a cyanoacrylate-based embolization substance and is capable of restraining the embolization substance injected into a blood vessel from instantaneously hardening so that a lesion site of the blood vessel can be embolized securely and safely. The blood vessel embolization method which achieves the above-described object is as described below.

It is another object of the present invention to provide a balloon catheter, for a blood embolization, to be used in the blood vessel embolization method. The balloon catheter for the blood vessel embolization method which achieves the above-described object is as described below.

### BRIEF DESCRIPTION OF THE DRAWINGS

FIG. 1 is a partly abbreviated appearance view of one embodiment of a balloon catheter for a blood vessel embolization method of the present invention.

FIG. 2 is an enlarged appearance view of a distal portion of the balloon catheter shown in FIG. 1.

FIG. 3 is an enlarged sectional view of the distal portion of the balloon catheter shown in FIG. 1.

FIG. 4 is an enlarged longitudinal view of a proximal portion of the balloon catheter shown in FIG. 1.

FIG. 5 is an enlarged appearance view of the distal portion of the balloon catheter shown in FIG. 1 when a bulged portion of the balloon catheter is stretched.

FIG. 6 is an explanatory view for explaining a blood vessel embolization method of a first embodiment of the present invention.

FIG. 7 is an explanatory view for explaining the blood vessel embolization method of the first embodiment of the present invention.

FIG. 8 is an explanatory view for explaining the blood vessel embolization method of the first embodiment of the present invention.

FIG. 9 is an explanatory view for explaining the blood vessel embolization method of the first embodiment of the present invention.

FIG. 10 is an explanatory view for explaining the blood vessel embolization method of the first embodiment of the present invention.

FIG. 11 is an explanatory view for explaining a blood vessel embolization method of a second embodiment of the present invention.

FIG. 12 is an explanatory view for explaining the blood vessel embolization method of the second embodiment of the present invention.

FIG. 13 is an explanatory view for explaining the blood vessel embolization method of the second embodiment of the present invention.

FIG. 14 is an explanatory view for explaining the blood vessel embolization method of the second embodiment of the present invention.

FIG. 15 is an explanatory view for explaining the blood vessel embolization method of the second embodiment of the present invention.

FIG. 16 is an explanatory view for explaining a blood vessel embolization method of a third embodiment of the present invention.

FIG. 17 is an explanatory view for explaining the blood vessel embolization method of the third embodiment of the present invention.

FIG. 18 is an explanatory view for explaining the blood vessel embolization method of the third embodiment of the present invention.

FIG. 19 is an explanatory view for explaining the blood vessel embolization method of the third embodiment of the present invention.

FIG. 20 is an explanatory view for explaining the blood vessel embolization method of the third embodiment of the present invention.

FIG. 21 is an explanatory view for explaining a blood vessel embolization method of a fourth embodiment of the present invention.

FIG. 22 is an explanatory view for explaining the blood vessel embolization method of the fourth embodiment of the present invention.

FIG. 23 is an explanatory view for explaining the blood vessel embolization method of the fourth embodiment of the present invention.

FIG. 24 is an explanatory view for explaining the blood vessel embolization method of the fourth embodiment of the present invention.

FIG. 25 is an explanatory view for explaining the blood vessel embolization method of the fourth embodiment of the present invention.

FIG. 26 is an explanatory view for explaining the blood vessel embolization method of the fourth embodiment of the present invention.

### DETAILED DESCRIPTION OF THE PREFERRED EMBODIMENTS

Embodiment of the balloon catheter of the present invention for a blood vessel embolization method is described below with reference to the drawings.

The balloon catheter of the present invention for the blood vessel embolization method is used in a blood vessel embolization method to be described below.

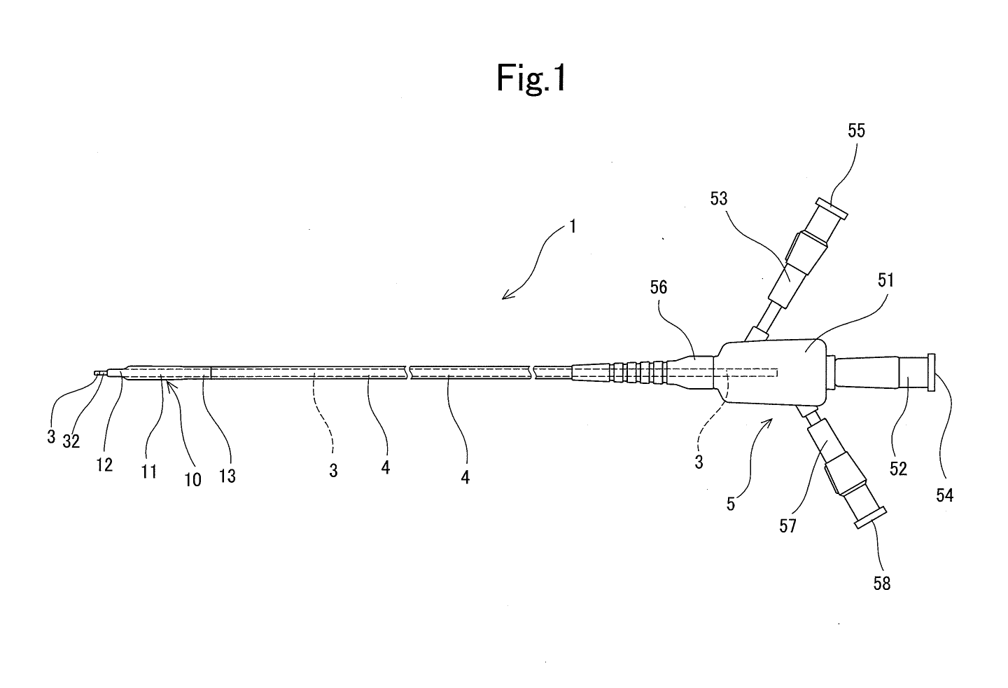

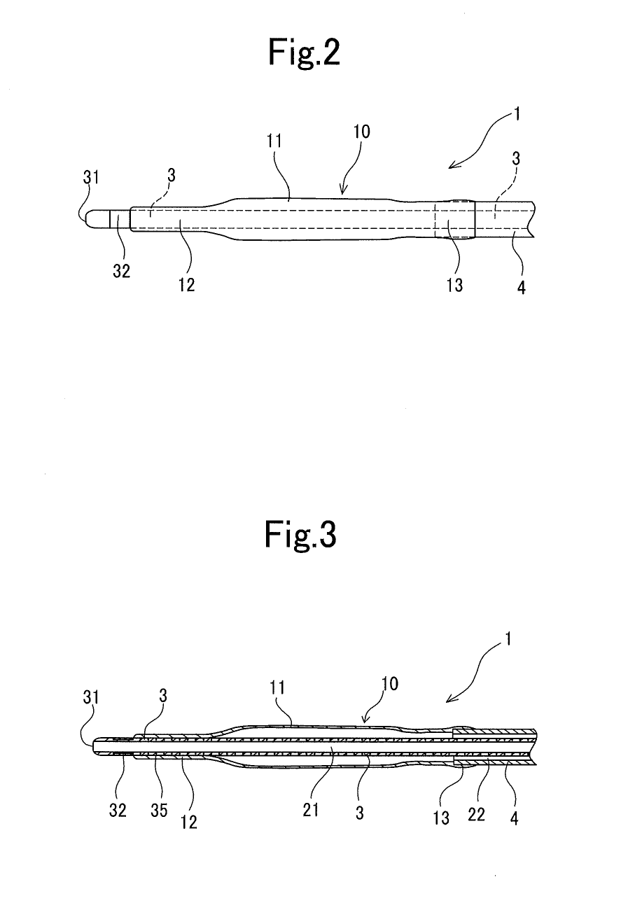

As shown in FIGS. 1 through 4, a balloon catheter 1 for the blood vessel embolization method has an inner tube 3 having a main lumen 21, an outer tube 4 provided coaxially with the inner tube 3 and forming a balloon expansion lumen 22 between an outer surface of the inner tube 3 and an inner surface thereof, a balloon 10 expandable and communicating with the balloon expansion lumen 22, and a hub 5 fixed to a proximal portion of the inner tube 3 and that of the outer tube 4.

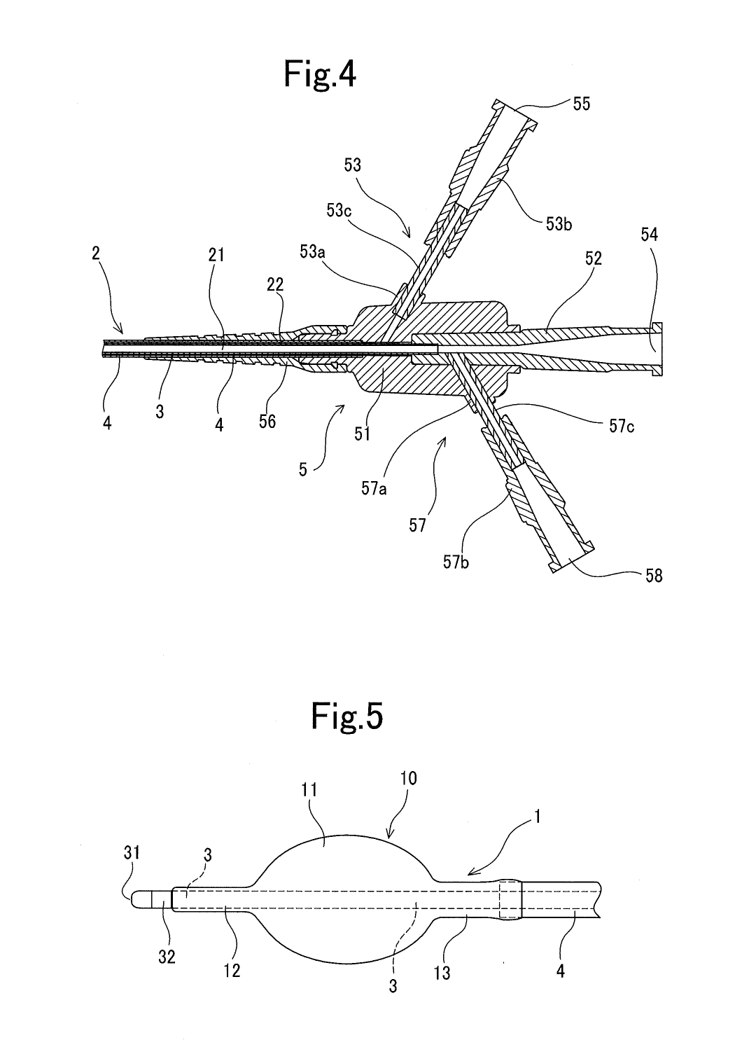

As shown in FIGS. 1 and 4, the hub 5 has a first port 52, linearly communicating with a proximal portion of the main lumen 21, to which a first liquid injection tool (not shown in the drawings) in which a glucose solution is filled can be connected, a second port 57, obliquely communicating with the main lumen 21 or the inside of the first port 52, to which a second liquid injection tool (not shown in the drawings) in which a cyanoacrylate-based embolization substance-containing liquid is filled can be connected, and a third port 53, communicating with the balloon expansion lumen 22, to which a liquid injection tool (not shown in the drawings) for injecting a balloon expansion fluid to the third port 53 can be connected.

In the balloon catheter 1 of the present invention for the blood vessel embolization method, because the branch hub 5 has the first and second ports 52, 57 communicating with the main lumen 21, it is possible to simultaneously connect the first liquid injection tool in which the glucose solution is filled and the second liquid injection tool in which the cyanoacrylate-based embolization substance-containing liquid is filled to the first and second ports 52 and 57 respectively.

The balloon catheter 1 of this embodiment is constructed of the inner tube 3, the outer tube 4, the balloon 10, and the hub 5.

The inner tube 3 is a tubular body having the main lumen 21 open at its distal end. The main lumen 21 is used to insert a guide wire into the balloon catheter and inject the glucose solution and the cyanoacrylate-based embolization substance-containing liquid to a blood vessel. In the balloon catheter 1 of this embodiment, the main lumen 21 of the inner tube 3 communicates with a first opening 54 and a second opening 58 both of which are formed on the branch hub 5.

The outer diameter of the inner tube 3 is favorably 0.60 to 1.63 mm and especially favorably 0.62 to 0.68 mm. The inner diameter thereof is favorably 0.40 to 1.33 mm and especially favorably 0.43 to 0.50 mm.

The inner tube 3 is inserted into the outer tube 4 such that its distal portion is projected beyond the outer tube 4. The balloon expansion lumen 22 is formed between the outer surface of the inner tube 3 and the inner surface of the outer tube 4 and has a sufficiently large volume.

An angiographic marker 32 is fixed to the distal portion (disposed a little proximally from the distal end 31 of the inner tube 3 and in the neighborhood of tubular distal part 12 of the balloon 10) of the inner tube 3. It is preferable to form the angiographic marker 32 of a radiopaque substance (for example, gold, platinum, tungsten, alloys thereof, silver-palladium alloy, and platinum-iridium alloy). By so doing, it is possible to confirm the distal portion of the balloon catheter 1 with the radiographic visualization.

The outer tube 4 is a tubular body into which the inner tube 3 is inserted. The distal end of the outer tube 4 is positioned at a portion (disposed at proximal side in a predetermined length) backward from the distal end of the inner tube 3. The distal end of the balloon expansion lumen 22 communicates with the proximal end of the balloon 10 to be described below. The proximal end of the balloon expansion lumen 22 communicates with a third opening 55 of the third port (injection port) 53, provided on the branch hub 5, into which a fluid (for example, balloon expansion liquid, namely, blood contrast agent) for expanding the balloon is injected.

The outer diameter of the outer tube 4 is favorably 0.85 to 2.03 mm and especially favorably 0.87 to 0.95 mm. The inner diameter thereof is favorably 0.70 to 1.83 mm and especially favorably 0.72 to 0.80 mm.

As materials of the outer tube 4 and the inner tube 3, materials having a certain degree of hardness and flexibility are preferable. For example, it is possible to use polyolefins such as polyethylene, polypropylene, and the like; polyamide, polyesters such as polyethylene terephthalate, and the like; fluorine-based polymers such as PTFE, ETFE, and the like; PEEK (polyether ether ketone); polyimide; olefin-based elastomers (for example, polyethylene elastomer, polypropylene elastomer); polyamide elastomers; styrene-based elastomers (for example, styrene-butadiene-styrene copolymer, styrene-isoprene-styrene copolymer, and styrene-ethylene butylene-styrene copolymer); synthetic resin elastomers such as polyurethane, urethane-based elastomers, and fluororesin-based elastomers; and synthetic rubbers such as urethane rubber, silicone rubber, and butadiene rubber; and rubber such as natural rubber, for example, Latex rubber.

It is possible to use the balloon 10 extendable, foldable, and extendable and foldable. In the catheter of this embodiment, the balloon which is allowed to have a state in which it is folded on the periphery of the inner tube 3, restored to a molded form by a balloon expansion fluid, and is extendable (expandable) is used.

As materials of the balloon 10, it is possible to use polyolefin, polyvinyl chloride, polyamide, polyester, polyarylene sulfide, synthetic resin elastomers such as polyamide elastomers, polyester elastomers, urethane-based elastomers, and rubber materials such as silicone rubber, Latex rubber, and the like. Polyurethane-based thermoplastic elastomers, for example, aromatic polyurethane-based thermoplastic elastomers and aliphatic polyurethane-based thermoplastic elastomers are especially preferable. As examples of the polyurethane-based thermoplastic elastomers, aromatic or aliphatic thermoplastic polyurethane elastomers are exemplified. The balloon 10 is more flexible than the outer tube 4. The balloon 10 more flexible than the inner tube 3 and the outer tube 4 is preferable.

The balloon 10 has an expandable part 11, a tubular distal part 12 extended toward the distal end thereof from the expandable part 11, and a tubular proximal part 13 extended toward the proximal end thereof from the expandable part 11.

The tubular distal part 12 of the balloon 10 is extended in a predetermined length from the distal end of the expandable part 11 and fixed to the distal portion of the inner tube 3. The outer diameter of the tubular distal part 12 of the balloon 10 is smaller than that of the tubular proximal part 13 disposed proximally from the expandable part 11. It is preferable that the distal end of the tubular distal part 12 of the balloon 10 is disposed at the proximal end of the angiographic marker 32 or at a position proximal from the proximal end thereof and close thereto. It is also preferable that the tubular distal part 12 of the balloon 10 does not cover the angiographic marker 32. It is also preferable to fix the tubular distal part 12 of the balloon 10 to the inner tube 3 by heat sealing.

The tubular proximal part 13 of the balloon 10 is extended in a predetermined length from the proximal end of the expandable part 11 and fixed to the distal portion of the outer tube 4. It is preferable to fix the tubular proximal part 13 of the balloon 10 to the outer tube 4 by heat sealing.

The expandable part 11 is expanded by a liquid injected thereinto and adhesive to the inner wall of a lumen (specifically, blood vessel). The tubular distal part 12 is short and extended in almost the same diameter. The tubular proximal part 13 is also short and extended in almost the same diameter. The inside of the expandable part 11 and that of the tubular proximal part 13 communicate with the balloon expansion lumen 22 formed between the outer surface of the inner tube 3 and the inner surface of the outer tube 4.

The outer diameter of the expandable part 11 of the balloon 10 when the expandable part 11 is expanded to the maximum is favorably 3.0 to 15.0 mm and especially favorably 4.0 to 8.0 mm. The length of the expandable part 11 is favorably 3.5 to 14.5 mm and especially favorably 4.0 to 5.5 mm. The outer diameter of the tubular distal part 12 is favorably 0.77 to 1.85 mm and especially favorably 0.80 to 0.90 mm. The length of the tubular distal part 12 is favorably 1.0 to 3.0 mm and especially favorably 1.5 to 2.5 mm. The outer diameter of the tubular proximal part 13 is favorably 0.90 to 2.10 mm and especially favorably 0.93 to 1.00 mm. The length of the tubular proximal part 13 is favorably 10 to 60 mm and especially favorably 15 to 30 mm.

The expandable part 11 of the balloon 10 is thinner than the tubular distal part 12 and the tubular proximal part 13 so that the expandable part 11 can be easily expanded and extended by an expansion liquid to be injected to the balloon 10. The thickness of the expandable part 11 is thinner than that of the tubular distal part 12 and that of the tubular proximal part 13 favorably by 0.03 to 0.18 mm and especially favorably by 0.04 to 0.11 mm. The thickness of the tubular distal part 12 and that of the tubular proximal part 13 are favorably 0.07 to 0.20 mm and especially favorably 0.08 to 0.15 mm.

As shown in FIG. 4, the branch hub 5 has the third port 53, serving as an injection port, which has a third opening 55 communicating with the balloon expansion lumen 22. The branch hub 5 has a first hub member 51 fixed to the proximal portion of the outer tube 4 and a second hub member which has the first opening 54 communicating with the main lumen 21, is fixed to the proximal portion of the inner tube 3, and forms the first port 52. The first hub member 51 has the second port 57 to which the second liquid injection tool is connectable. The second port 57 branches from the first hub member 51, penetrates through the first port 52, and communicates with the inside of the first port 52, thus communicating with the main lumen 21. The first hub member 51 and the second hub member (first port) 52 are fixed to each other. The hub 5 has a bending prevention tube 56 enclosing the proximal portion of the outer tube 4 and the distal portion of the hub 5.

The third port 53 into which the balloon expansion fluid is to be injected is constructed of a lateral opening 53a having a passage extended from a side wall of the first hub member 51 and communicating with the inside of the first hub member 51, a port hub 53b to which the liquid injection tool is connectable, and a connection tube 53c connecting the cylindrical opening 53a and the port hub 53b to each other. The connection tube 53c enters into the lateral opening 53a and is fixed to the first hub member 51.

The second port 57 to which the second liquid injection tool is connectable is constructed of a lateral opening 57a penetrating through a side wall of the first hub member 51 and reaching a side surface of the second hub member (first port) 52, a port hub 57b to which the liquid injection tool is connectable, and a connection tube 57c connecting the lateral opening 57a and the port hub 57b to each other. The connection tube 57c penetrates through the lateral opening 57a and enters into an opening formed through the side surface of the second hub member 52. Thus the second port 57 communicates with the first port 52, thereby communicating with the main lumen 21. The connection tube 57c is fixed to the first hub member 51 and liquid-tightly fixed to the first port 52.

As a material of the branch hub 5, it is possible to preferably use thermoplastic resins such as polycarbonate, polyamide, polysulfone, polyarylate, and a methacrylate-butylene-styrene copolymer. As the connection tube 57c, tubes made of flexible or soft synthetic resin can be used.

The blood vessel embolization method of the present invention to be carried out by using the balloon catheter is described above with reference to the drawings.

The blood vessel embolization method of the present invention is carried out by using the balloon catheter having a main lumen and a balloon expansion lumen.

The blood vessel embolization method of the present invention includes: a balloon catheter insertion step of inserting the balloon catheter into a blood vessel of a human or an animal having a lesion site and disposing a balloon of the balloon catheter at a portion located at a proximal side of the blood vessel and in a neighborhood of the lesion site thereof or at a portion not spaced at a long distance from the lesion site and disposed at the proximal side of the blood vessel; a blood flow shut-off step of shutting off a blood flow in the blood vessel by injecting a fluid into the balloon expansion lumen to expand the balloon; a glucose solution injection step of discharging a glucose solution from a distal end of the balloon catheter by using the main lumen of the balloon catheter in a state in which the blood flow inside the blood vessel is shut off; an embolization substance injection step of discharging a cyanoacrylate-based embolization substance-containing liquid from the distal end of the balloon catheter by using the main lumen of the balloon catheter after the glucose solution injection step (in other words, after the injection of the glucose solution finishes); an embolization substance-hardening step of hardening a embolization substance by maintaining a blood flow shut-off state of the blood vessel after the embolization substance injection step (in other words, after the discharge of the embolization substance finishes); and a balloon catheter removal step of removing the balloon catheter from the blood vessel after the balloon is contracted after the embolization substance-hardening step.

The blood vessel embolization method of the present invention is applicable to all blood vessels to be embolized. As blood vessels to be embolized, it is possible to list trauma-caused bleeding, bleeding caused by rupture of tumors, bleeding caused by blood vessel diseases (aneurysm rupture, blood vessel malformation), advance occlusion of blood vessel diseases (aneurysm rupture, blood vessel malformation), blood vessels of the abdominal organs having lesion sites (for example, tumor) typically the artery in the liver, the lineal artery, the renal artery, the internal iliac artery, the arteries of four limbs, and branching blood vessels of these arteries, and further the lumbar artery and the intercostals artery. A procedure include an embolization method for bleeding from external injury (liver, pancreas, spleen, kidney, and pelvic bone), tumors, and the digestive tract, an embolization method for arteries of tumor sites such as hepatocellular tumor, kidney cancer, and the like; an embolization method for bronchial arteries which have developed hemoptysis; a preoperative embolization method for plethoric bone metastasis (the intercostals artery and the lumbar artery are embolized to decrease the amount of bleeding during an operation before performing an operation of bony metastasis from tumors such as hepatocellular tumor, kidney cancer, and the like where much blood circulates); and an embolization method for artery of uterus having fibroid. The blood vessel embolization method is particularly effective for embolizing the artery including the splenic artery, the kidney artery, the liver artery, the intercostal artery, branching of a mesenteric artery, the lumbar artery, the gastroduodenal artery, and the inner and outer iliac arteries.

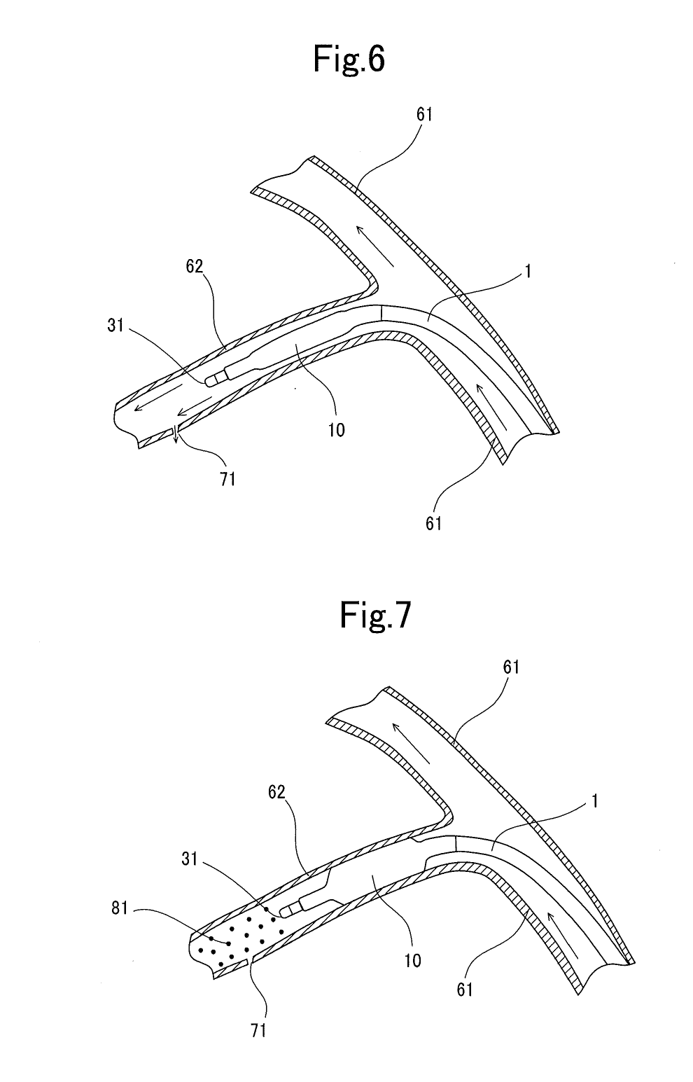

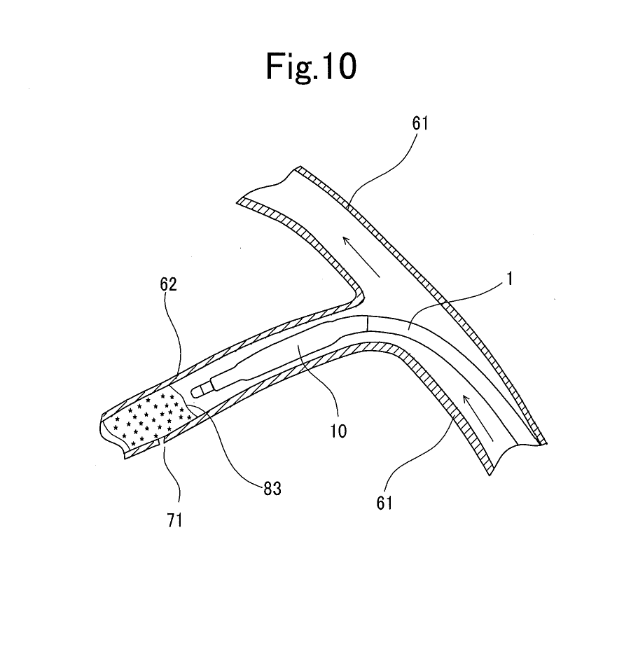

One embodiment of a first blood vessel embolization method of the present invention is described below with reference to FIGS. 6 through 10.

In this blood vessel embolization method, only the balloon catheter is used as the catheter. The blood vessel embolization method of this embodiment is different from a blood vessel embolization method to be described later in that in this embodiment, a tubular catheter (for example, microcatheter) is not used inside the balloon catheter. As the balloon catheter, it is preferable to use a microballoon catheter in which the outer diameter of the outer tube is not more than 1.20 mm.

A case shown in FIG. 6 has a hemorrhagic lesion site 71 spaced a little distally from a branching portion of a branching blood vessel 62 branching from an artery (for example, artery in liver) 61. The lesion site is identified by an angiographic method in which an angiographic catheter and a contrast medium are used. In an angiography, a guide wire is inserted into a Seldinger needle pierced into a femoral artery to insert the guide catheter (not shown in the drawings) into the neighborhood of a branching portion, of the artery in the liver, having the lesion site under the guide of the guide wire.

The balloon catheter 1 which can be inserted into the guide catheter is prepared. A case in which the balloon catheter 1 is used is described above as an example.

Initially the balloon catheter insertion step is performed. At this step, as shown in FIG. 6, after the balloon catheter 1 is inserted into the branching blood vessel 62 of a human or an animal having the lesion site 71, the balloon 10 of the balloon catheter 1 is disposed at a portion (the portion shown in FIG. 6 is disposed a little proximally from the lesion site of the branching blood vessel) at a proximal side of the branching blood vessel 62 in the neighborhood of the lesion site thereof. The lesion site 71 is normally a bleeding site. The balloon 10 is located at a portion proximal from the bleeding site of the branching blood vessel 62 and at the proximal side of the branching blood vessel 62 in the neighborhood of the branching portion. That is, as shown in FIG. 6, the balloon catheter 1 is so disposed that the distal end thereof is spaced slightly proximally from the lesion site 71 and that the balloon 10 is disposed inside the branching blood vessel 62 having the lesion site 71.

Thereafter the blood flow shut-off step of shutting off a blood flow in the blood vessel by injecting a fluid into the balloon expansion lumen to expand the balloon is performed. More specifically, in a state in which the balloon 10 is positioned in the branching blood vessel 62 having the lesion site 71, a syringe (not shown in the drawings) in which an expansion liquid (for example, radiographic contrast medium) is filled is connected to the third port 53 to which a fluid for expanding the balloon 10 of the balloon catheter 1 is to be injected. Thereafter a plunger of the syringe is pressed to inject the radiographic contrast medium into the balloon 10 from the lumen 22 so that the balloon 10 is expanded, as shown in FIG. 7. Thereby the blood flow in the branching blood vessel 62 having the lesion site 71 is shut off. The balloon catheter 1 is fixed to the branching blood vessel 62. An expanded state of the balloon 10, in other words, a state in which the balloon catheter 1 has been fixed to the inside of the balloon catheter 1 can be confirmed by the radiographic visualization.

Thereafter the glucose solution injection step is performed. At the glucose solution injection step, a glucose solution is discharged from the distal end of the balloon catheter 1 by using the main lumen 21 of the balloon catheter 1 in a state in which the blood flow inside the branching blood vessel 62 having the lesion site 71 is shut off. At the glucose solution injection step, the glucose solution injection step is performed so that a discharged glucose solution arrives at the lesion site 71. Also, at the glucose solution injection step, the glucose solution is filled in a region, of the branching blood vessel 62, which is disposed between the distal end of the expanded balloon 10 and the lesion site 71 of the branching blood vessel 62. As shown in FIG. 7, as a result of the injection of the glucose solution into the above-described region, the glucose solution is filled in the branching blood vessel 62 in the region from the distal end of the balloon catheter 1 to a portion spaced distally from the lesion site 71. At the portion of the branching blood vessel 62 where the glucose solution has been filled, a considerable amount of blood is pressed out to the distal side of the branching blood vessel 62. Thus even though a small amount of blood remains in the branching blood vessel 62, the blood is considerably diluted.

At the glucose solution injection step, it is preferable to use the first port 52 disposed at the proximal portion of the balloon catheter 1 and linearly communicating with the main lumen 21. By so doing, when the glucose solution injection step finishes, the glucose solution is filled inside the main lumen 21 and the first port 52. As the glucose solution, 5% glucose-containing water is preferable. After the syringe (not shown in the drawings) in which the glucose solution has been filled is connected to the opening 54 of the first port 52, the glucose solution is discharged into the first port 52 from the opening 54. In a normal case, the injection amount of the glucose solution is preferably 1 to 3 ml. In the case where the guide wire is inserted into the balloon catheter 1 before the glucose solution injection step is performed, the guide wire is removed therefrom.

In the blood vessel embolization method of the present invention, it is preferable to perform an interval confirming step of confirming an approximate interval between the lesion site 71 and the distal end of the balloon catheter 1 and a glucose solution injection amount decision step of deciding the amount of the glucose solution to be injected at the glucose solution injection step in consideration of the approximate interval confirmed at the interval confirming step. After the blood flow shut-off step of shutting off the blood flow in the blood vessel having the lesion site 71 by expanding the balloon 10 finishes, the interval confirming step can be accomplished by injecting the contrast medium from the main lumen 21 of the balloon catheter 1 and confirming the interval between the distal end of the balloon catheter 1 and the lesion site 71 by means of the radiographic visualization. The injection amount (discharge amount) of the glucose solution is decided in consideration of the diameter of the branching blood vessel 62 and the interval between the distal end of the balloon catheter 1 and the lesion site 71. The injection amount of the glucose solution may be decided by using a value obtained by calculation in consideration of the interval between the distal end of the balloon catheter 1 and the lesion site 71 and the diameter of the branching blood vessel 62.

In the blood vessel embolization method of the present invention, a determination step of determining whether the interval between the distal end of the balloon catheter 1 and the lesion site is more than a predetermined distance may be performed. When it is determined at the determination step that the interval between the distal end of the balloon catheter 1 and the lesion site is not more than the predetermined distance, a first discharge amount of the glucose solution is discharged at the glucose solution injection step. When it is determined at the determination step that the interval between the distal end of the balloon catheter and the lesion site is more than the predetermined distance, it is preferable to discharge a second discharge amount of the glucose solution which is more than the first discharge amount.

The determination step can be accomplished by injecting the contrast medium into the region between the distal end of the balloon catheter 1 and the lesion site 71 from the main lumen 21 of the balloon catheter 1 and confirming the interval between the distal end of the balloon catheter 1 and the lesion site 71 by the radiographic visualization, after the blood flow shut-off step of shutting off the blood flow in the blood vessel having the lesion site 71 by expanding the balloon 10 finishes. When it is determined at the determination step that the interval between the distal end of the balloon catheter and the lesion site is not more than the predetermined interval, the first discharge amount of the glucose solution decided in consideration of the diameter of the blood vessel is discharged. When it is determined at the determination step that the interval between the distal end of the balloon catheter and the lesion site is more than the predetermined interval, the second discharge amount of the glucose solution which is more than the first discharge amount decided in consideration of the diameter of the blood vessel is discharged.

Thereafter the embolization substance injection step is performed. At the embolization substance injection step, the cyanoacrylate-based embolization substance-containing liquid is discharged from the distal end of the balloon catheter 1 by using the main lumen 21 of the balloon catheter 1, after the injection of the glucose solution finishes. At the embolization substance injection step, the cyanoacrylate-based embolization substance-containing liquid is injected into the region, between the distal end of the balloon catheter 1 and the lesion site 71 of the branching blood vessel 62, in which the blood diluted by the injected glucose solution has been filled such that the cyanoacrylate-based embolization substance-containing liquid flows toward the lesion site 71.

More specifically, at the embolization substance injection step, it is preferable to connect the syringe (not shown in the drawings) in which the cyanoacrylate-based embolization substance-containing liquid has been filled to the second opening 58 of the third port 53 which is the second branching portion disposed at the proximal portion of the hub 5 of the balloon catheter 1 and obliquely communicating with the main lumen (specifically, first port 52) 21. By so doing, when the glucose injection step finishes, the cyanoacrylate-based embolization substance-containing liquid is injected into the glucose solution filled inside the main lumen 21 or the port 52. Thereby the cyanoacrylate-based embolization substance-containing liquid does not harden inside the catheter. The syringe in which the cyanoacrylate-based embolization substance-containing liquid has been filled may be connected to the second opening 58 of the third port 53 before the operation of discharging the glucose solution is performed at the glucose solution injection step.

As the cyanoacrylate-based embolization substance, liquid N-butyl-cyanoacrylate is preferable. It is preferable that the cyanoacrylate-based embolization substance-containing liquid contains a contrast medium substance. As the contrast medium substance, a liquid radiographic contrast medium is preferable. As the liquid radiographic contrast medium, an ethyl ester solution of iodized fatty acid of poppy seed oil is preferable. Therefore as the cyanoacrylate-based embolization substance-containing liquid, it is preferable to use a mixture of the liquid N-butyl-cyanoacrylate and the ethyl ester solution of iodized fatty acid of poppy seed oil. The mixing ratio between the liquid N-butyl-cyanoacrylate and the ethyl ester solution of iodized fatty acid of poppy seed oil is preferably 1:2 to 2:1.

In a normal case in which the blood vessel is embolized, the injection amount of the cyanoacrylate-based embolization substance-containing liquid is preferably 0.1 to 0.3 ml. In the case where the interval confirming step is performed, it is preferable to perform an embolization substance-containing liquid injection amount decision step of deciding the injection amount of the cyanoacrylate-based embolization substance-containing liquid to be injected at the embolization substance injection step in consideration of the approximate distance confirmed at the interval confirming step.

It is preferable to slowly inject the cyanoacrylate-based embolization substance-containing liquid into the predetermined region. By slowly injecting the cyanoacrylate-based embolization substance-containing liquid into the predetermined region, it is possible to prevent the cyanoacrylate-based embolization substance-containing liquid from collecting on the distal-end surface of the balloon in such a way that it lies thereon. The injection speed of the cyanoacrylate-based embolization substance-containing liquid is preferably 0.1 ml/1 to 3 seconds. When the cyanoacrylate-based embolization substance-containing liquid contains a liquid radiographic contrast medium, the arrival thereof at the lesion site 71 can be confirmed by the radiographic visualization.

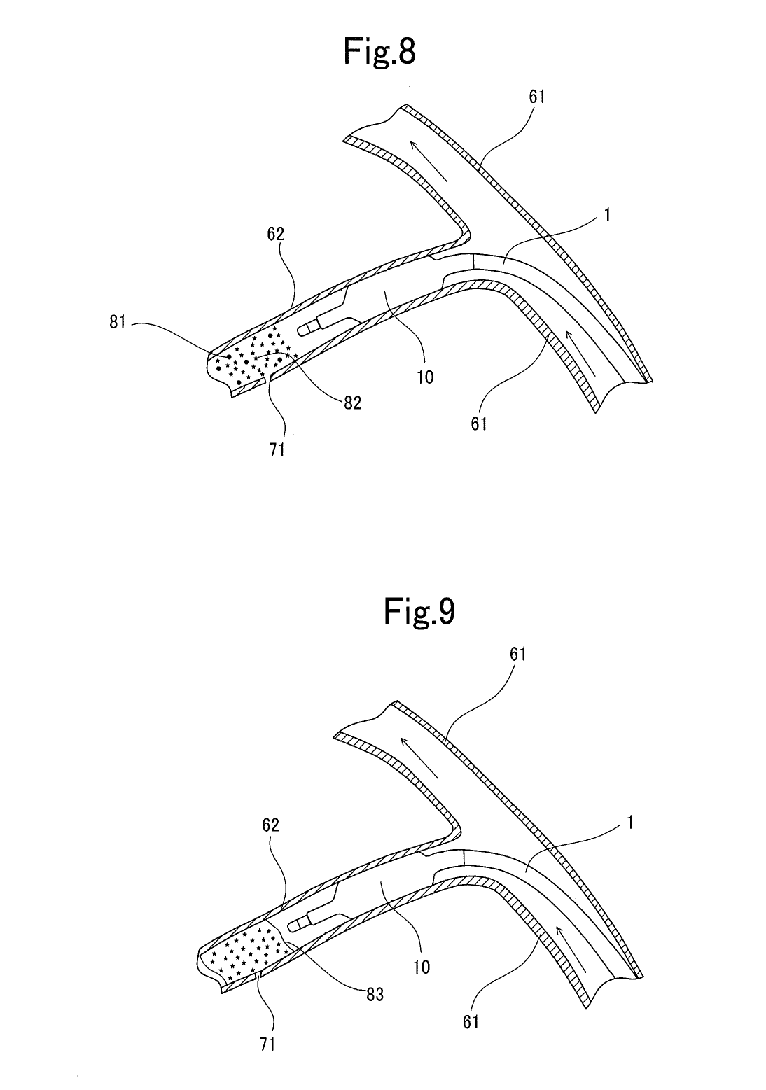

The glucose solution injected into the above-described predetermined region at the glucose solution injection step inhibits the hardening of the cyanoacrylate-based embolization substance. Exactly describing, the cyanoacrylate-based embolization substance contacts cations (calcium ion, sodium ion, and the like) in the blood, thus polymerizing and hardening. As shown in FIG. 8, a cyanoacrylate-based embolization substance-containing liquid 82 discharged from the distal end of the balloon catheter 1 at the glucose solution injection step is injected into the predetermined region of the branching blood vessel 62 in which a glucose solution 81 has been filled. The presence of the glucose solution in the blood restricts contact between the cyanoacrylate-based embolization substance and the cations in the blood, thus delaying the hardening of the cyanoacrylate-based embolization substance. Therefore the cyanoacrylate-based embolization substance-containing liquid does not harden immediately after the cyanoacrylate-based embolization substance-containing liquid is injected into the above-described predetermined region, but can be continuously injected to the predetermined region until it reaches the lesion site 71 to be embolized.

Thereafter the embolization substance-hardening step is performed. The embolization substance-hardening step is performed by maintaining a blood flow shut-off state (in other words, expanded state of the balloon 10) of the branching blood vessel having the lesion site 71 and leaving the shut-off state for a predetermined period of time, after the discharge of the cyanoacrylate-based embolization substance-containing liquid finishes. The glucose solution diffuses gradually. Thereby the contact between the cyanoacrylate-based embolization substance and the cations in the blood progresses. As a result, the cyanoacrylate-based embolization substance hardens. The hardened cyanoacrylate-based embolization substance forms a embolized site 83 at a portion which is disposed in the neighborhood of the lesion site 71 to be embolized and at the proximal side of the branching blood vessel 62, as shown in FIG. 9. Thereby the branching blood vessel 62 having the lesion site 71 to be embolized is shut off. The glucose solution filled in the predetermined region at the glucose solution injection step prevents the connection between the distal end of the balloon catheter 1 and the embolized site 83.

Thereafter the balloon catheter removal step is performed. At the balloon catheter removal step, after the embolization substance-hardening step finishes, the balloon catheter 1 is removed from the blood vessel after the balloon 10 is contracted.

To harden the cyanoacrylate-based embolization substance more reliably, in other words, to form the perfect embolized site, after the embolization substance-hardening step is performed by keeping a state in which the balloon 10 is expanded, an auxiliary embolization substance hardening step of circulating a small amount of blood to the embolized site 83 may be performed by contracting and expanding (in other words, pumping) the balloon 10 once or plural times by keeping the state in which the balloon catheter 1 has been disposed.

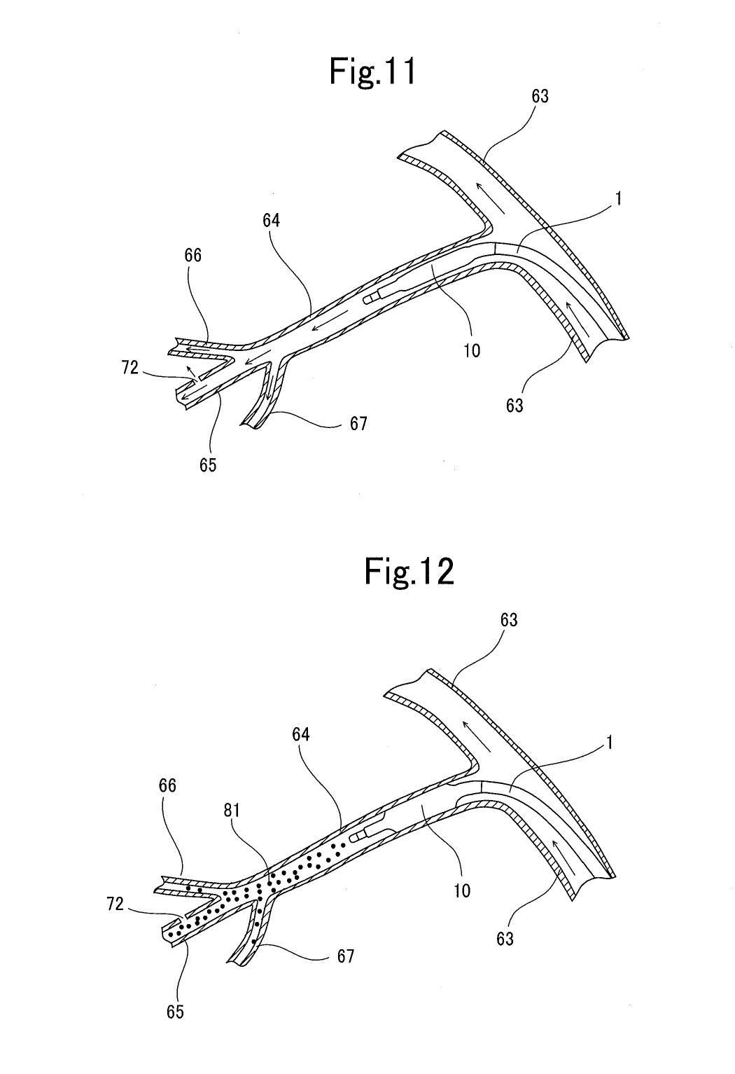

Another embodiment of the first blood vessel embolization method of the present invention is described below with reference to FIGS. 11 through 15.

Only the balloon catheter is used as the catheter in the blood vessel embolization method of this embodiment. The blood vessel embolization method of this embodiment is different from a blood vessel embolization method to be described later in that in the former, a catheter is not used inside the balloon catheter.

In a case shown in FIG. 11, a hemorrhagic lesion site 72 is generated in a blood vessel 65 of blood vessels 65, 66, and 67 branching from a branching blood vessel 64 branching from an artery (for example, artery in liver) 63. The lesion site 72 is identified by an angiographic method in which the angiographic catheter and the contrast medium are used. In this case, because the branching blood vessel 65 having the lesion site 72 has a small diameter, it is impossible to insert the balloon 10 of the balloon catheter 1 into the branching blood vessel 65. The balloon catheter insertion step, the glucose solution injection step, the embolization substance injection step, the embolization substance hardening step, and the balloon catheter removal step are performed in a manner similar to that described above respectively. As the balloon catheter, it is preferable to use a microballoon catheter in which the outer diameter of the outer tube is not more than 1.20 mm.

The balloon catheter insertion step is performed as follows: At this step, as shown in FIG. 11, the balloon catheter 1 is inserted into the branching blood vessel 64 communicating with the branching blood vessel 65 which has the lesion site 72 and is disposed proximally from the branching blood vessel 65 in such a way that the balloon 10 of the balloon catheter 1 is disposed at the portion proximal (a portion disposed proximally from the lesion site of the branching blood vessel 65 and spaced in a predetermined length from lesion site) from the branching portion where the branching blood vessel 65 branches from the branching blood vessel 64.

Thereafter the blood flow shut-off step of shutting off the blood flow in the blood vessel having the lesion site is performed by injecting a fluid into the balloon expansion lumen to expand the balloon 10. More specifically, the radiographic contrast medium is injected into the balloon 10 to expand balloon 10, as shown in FIG. 12. Thereby the branching blood vessel 64 is closed. Thus the blood flow in the branching blood vessel 64 is shut off. Therefore the blood flow in the branching blood vessel 65, having the lesion site 72, which is disposed distally from the blood vessel 64 and that in the other branching blood vessels 66 and 67 also disposed distally from the blood vessel 64 are shut off. The balloon catheter 1 is fixed to the blood vessel 64.

Thereafter the glucose solution injection step is performed. At the glucose solution injection step, the glucose solution is discharged from the distal end of the balloon catheter 1 by using the main lumen 21 in a state in which the blood flow in the branching blood vessel 64 is shut off. The glucose solution injection step is performed so that a discharged glucose solution arrives at the lesion site 72. Also, As shown in FIG. 12, in the glucose solution injection step, the glucose solution is filled in the range from the distal end of the balloon 10 to the lesion site 72 of the branching blood vessel 65. In this case, the injected glucose solution flows not only into the branching blood vessel 65 having the lesion site 72, but also into the other branching blood vessels 66 and 67. It is preferable that at the glucose solution injection step, the glucose solution is injected into the first port 52 from the opening 54 thereof linearly communicating with the main lumen 21 of the balloon catheter 1.

As shown in FIG. 12, as a result of the injection of the glucose solution, the glucose solution is filled inside the branching blood vessel 65 in the range from the distal end of the balloon catheter 1 to the position disposed distally from the lesion site 72 and inside the branching blood vessels 66 and 67 as far as positions thereof disposed distally in predetermined length from the branching portion thereof. In portions of the branching blood vessels 65, 66, and 67 into which the glucose solution has been injected, the glucose solution forces a considerable amount of blood to the distal sides thereof. Even though a small amount of blood remains there, the blood is diluted to a high extent.

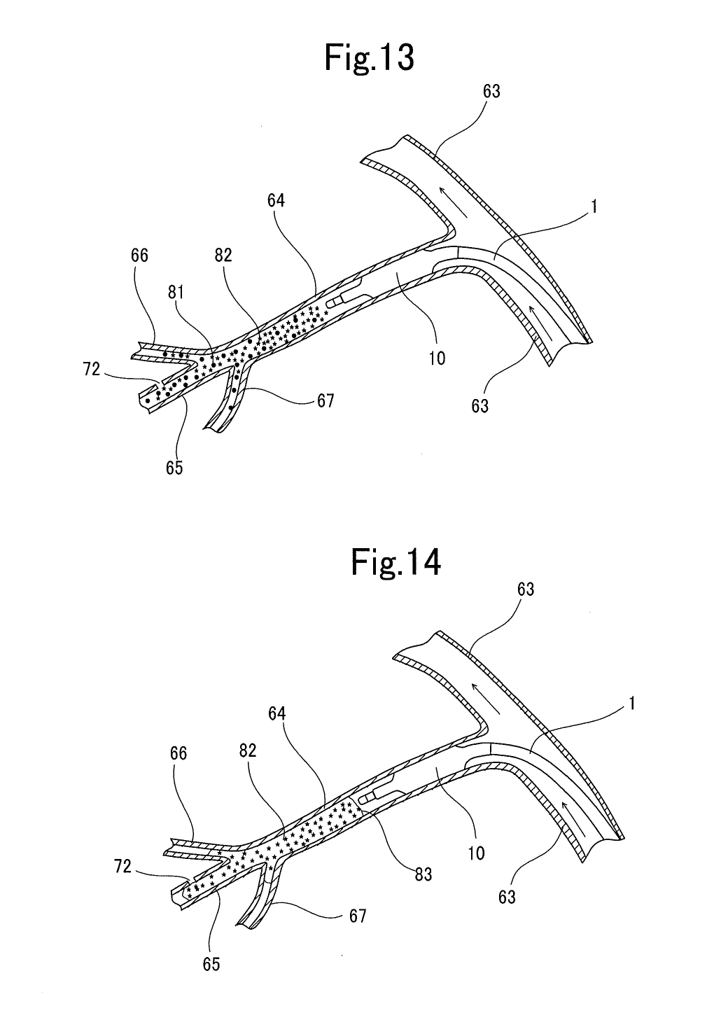

Thereafter the embolization substance injection step is performed. At the embolization substance injection step, the cyanoacrylate-based embolization substance-containing liquid is discharged from the distal end of the balloon catheter 1 by using the main lumen 21 of the balloon catheter 1, after the glucose solution is injected into the above-described predetermined region. At the embolization substance injection step, the cyanoacrylate-based embolization substance-containing liquid is injected into the region, between the distal end of the balloon catheter 1 and the lesion site 72 of the branching blood vessel 65, in which the blood diluted by the injected glucose solution has been filled such that the cyanoacrylate-based embolization substance-containing liquid flows toward the lesion site 71. As shown in FIG. 13, a cyanoacrylate-based embolization substance-containing liquid 82 discharged from the distal end of the balloon catheter 1 at the embolization substance injection step is injected into the blood vessel in which a glucose solution 81 has been filled. The presence of the glucose solution in the blood restricts contact between the cyanoacrylate-based embolization substance and cations in the blood, thus delaying the hardening of the cyanoacrylate-based embolization substance. Therefore the cyanoacrylate-based embolization substance-containing liquid does not harden immediately after the cyanoacrylate-based embolization substance-containing liquid is injected into the above-described predetermined region, but the cyanoacrylate-based embolization substance-containing liquid can be continuously injected to the predetermined region until it reaches the lesion site 71 to be embolized. It is preferable that at the glucose solution injection step, the glucose solution is injected into the third port obliquely communicating with the main lumen 21 (specifically, communicating with the inside of the first port 52) of the balloon catheter 1.

Thereafter the embolization substance-hardening step is performed. The embolization substance-hardening step is performed by keeping a blood flow shut-off state (in other words, expanded state of the balloon 10) and leaving the blood flow shut-off state for a predetermined period of time, after the discharge of the cyanoacrylate-based embolization substance-containing liquid finishes. The glucose solution diffuses gradually. Thereby the contact between the cyanoacrylate-based embolization substance and the cations in the blood progresses. As a result, the cyanoacrylate-based embolization substance hardens. The hardened cyanoacrylate-based embolization substance forms the embolized site 83 in the blood vessel 64 including the branching blood vessel 65 having the lesion site 72 to be embolized and embolizes the branching blood vessel 64, as shown in FIG. 14.

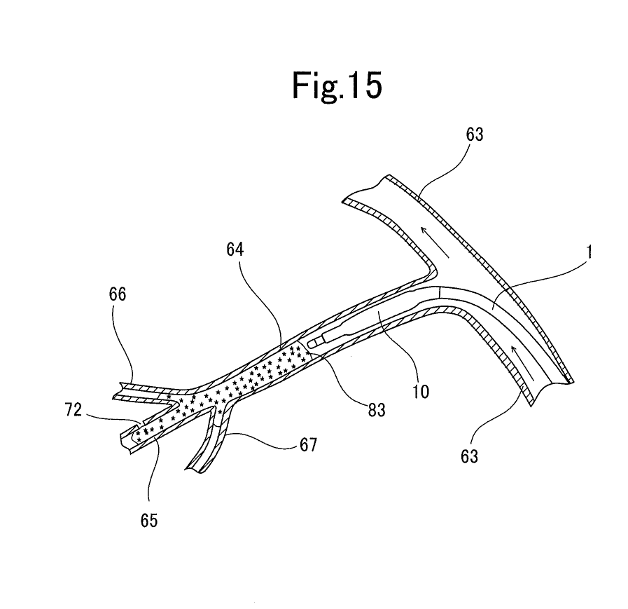

Thereafter the balloon catheter removal step is performed. As shown in FIG. 15, at the balloon catheter removal step, after the embolization substance-hardening step finishes, the balloon catheter 1 is removed from the blood vessel after the balloon 10 is contracted.

To harden the cyanoacrylate-based embolization substance more reliably, in other words, to form the perfect embolized site, after the embolization substance-hardening step is performed by keeping the state in which the balloon 10 is expanded, an auxiliary embolization substance hardening step of circulating a small amount of blood to the embolized site 83 may be performed by contracting and expanding (in other words, pumping) the balloon 10 once or plural times by keeping the state in which the balloon catheter 1 has been disposed.

In the blood vessel embolization method of this embodiment, it is preferable to perform the interval confirming step of confirming an approximate interval between the lesion site 72 and the distal end of the balloon catheter 1 and the glucose solution injection amount decision step of deciding the amount of the glucose solution to be injected at the glucose solution injection step in consideration of the above-described approximate distance and the other branching blood vessels 66 and 67 confirmed at the interval confirming step. After the blood flow shut-off step of shutting off the blood flow in the blood vessel having the lesion site 72 by expanding the balloon 10 finishes, the interval confirming step can be accomplished by injecting the contrast medium from the main lumen 21 of the balloon catheter 1 and confirming the interval between the distal end of the balloon catheter 1 and the lesion site 72 by means of the radiographic visualization. The injection amount (discharge amount) of the glucose solution is decided in consideration of the diameters of the branching blood vessels 64, 65, 66, and 67 and the interval between the distal end of the balloon catheter 1 and the lesion site 72. The injection amount of the glucose solution may be decided by using a value obtained by calculation in consideration of the interval between the distal end of the balloon catheter and the lesion site and the diameters of the branching blood vessels confirmed by an angiographic method. At the embolization substance injection step, it is preferable to decide the injection amount of the cyanoacrylate-based embolization substance-containing liquid in consideration of the approximate distance between the lesion site 72 and the distal end of the balloon catheter 1 confirmed at the interval confirming step.

In the blood vessel embolization method of this embodiment, the determination step of determining whether the interval between the distal end of the balloon catheter 1 and the lesion site 71 is more than the predetermined distance may be performed, as described in the above-described embodiment. When it is determined at the determination step that the interval between the distal end of the balloon catheter 1 and the lesion site is not more than the predetermined distance, the first discharge amount of the glucose solution is discharged at the glucose solution injection step. When it is determined at the determination step that the interval between the distal end of the balloon catheter 1 and the lesion site is more than the predetermined distance, it is preferable to discharge the second discharge amount of the glucose solution which is more than the first discharge amount.

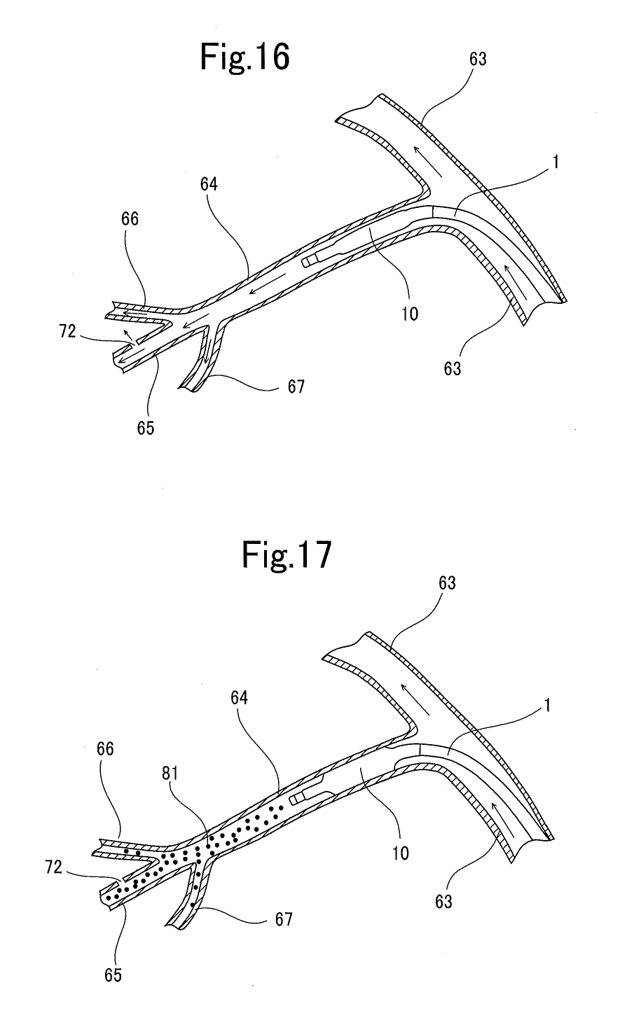

One embodiment of a second blood vessel embolization method of the present invention is described below with reference to FIGS. 16 through 20.

The blood vessel embolization method of this embodiment is carried out by using a balloon catheter having a main lumen and a balloon expansion lumen and a tubular catheter insertable into the balloon catheter.

The blood vessel embolization method of the present invention includes: a balloon catheter insertion step of inserting the balloon catheter into a blood vessel of a human or an animal having a lesion site and disposing a balloon of the balloon catheter at a portion located at a proximal side of the blood vessel and in a neighborhood of the lesion site thereof or at a portion not spaced at a long distance from the lesion site and disposed at the proximal side of the blood vessel; a tubular catheter insertion step of inserting the tubular catheter into the balloon catheter and locate a distal portion of the tubular catheter at a position close to the lesion site; a blood flow shut-off step of shutting off a blood flow in the blood vessel by injecting a fluid into the balloon expansion lumen to expand the balloon; a glucose solution injection step of discharging a glucose solution from a distal end of the balloon catheter in a state in which the blood flow inside the blood vessel is shut off; an embolization substance injection step of discharging a cyanoacrylate-based embolization substance-containing liquid from a distal end of the tubular catheter after the glucose solution injection step (after the injection of the glucose solution finishes); an embolization substance-hardening step of hardening a embolization substance by maintaining a blood flow shut-off state of the blood vessel after the embolization substance-hardening step (after the discharge of the embolization substance finishes); and a catheters removal step, including contraction of the balloon, of removing the balloon catheter and the tubular catheter from the blood vessel after the embolization substance-hardening step finishes.

It is preferable that the outer diameter of an outer tube of the balloon catheter to be used is not more than 3.50 mm. It is preferable that the catheter to be used has an outer diameter of not more than 2.40 mm and can be inserted into the balloon catheter. As described above, it is preferable that the cyanoacrylate-based embolization substance to be used consists of the N-butyl-cyanoacrylate. The above-described cyanoacrylate-based embolization substance-containing liquid can be preferably used.

The balloon catheter insertion step, the blood flow shut-off step of shutting off the blood flow in the blood vessel by injecting the fluid into the balloon expansion lumen to expand the balloon, and the glucose solution injection step of discharging the glucose solution from the distal end of the balloon catheter in the state in which the blood flow in the blood vessel is shut off are identical to those described in the above-described blood vessel embolization method. In the blood vessel embolization method of the present invention, it is preferable to use the balloon catheter 1 having the three ports as shown in FIGS. 1 through 5.

In a case shown in FIG. 16, the hemorrhagic lesion site 72 is generated in a blood vessel 65 of the blood vessels, 65, 66, and 67 branching from the branching blood vessel 64 branching from the artery (for example, artery in the liver) 63. The lesion site 72 is identified by the angiographic method in which the angiographic catheter and the contrast medium are used. In this case, because the branching blood vessel 65 having the lesion site 72 has a small diameter, it is impossible to insert the balloon 10 of the balloon catheter 1 into the branching blood vessel 65. The balloon catheter insertion step is performed as follows: At this step, as shown in FIG. 11, the balloon catheter 1 is inserted into the branching blood vessel 64 communicating with the branching blood vessel 65 which has the lesion site 72 and is disposed proximally from the branching blood vessel 65 in such a way that the balloon 10 of the balloon catheter 1 is disposed at the portion proximal (a portion disposed proximally from the lesion site of the branching blood vessel 65 and spaced in a predetermined length from lesion site) from the branching portion where the branching blood vessel 65 branches from the branching blood vessel 64. At the position where the balloon 10 is disposed, the blood flow shut-off step of shutting off the blood flow in the blood vessel by injecting the fluid into the balloon expansion lumen to expand the balloon is performed.

Thereafter the glucose solution injection step is performed by using the main lumen 21 of the balloon catheter. The glucose solution injection step is performed so that a discharged glucose solution arrives at the lesion site 72. Also, as shown in FIG. 17, at the glucose solution injection step, the glucose solution is filled in the region from the distal end of the expanded balloon 10 to the lesion site 72 of the branching blood vessel 65. In this case, the injected glucose solution flows not only into the branching blood vessel 65 having the lesion site 72 but also into the other branching blood vessels 66 and 67.

In the blood vessel embolization method of this embodiment, it is preferable to perform the interval confirming step of confirming an approximate interval between the lesion site 72 and the distal end of the balloon catheter 1, the glucose solution injection amount decision step of deciding the injection amount of the glucose solution to be injected at the glucose solution injection step in consideration of the approximate interval and the branching blood vessels 66 and 67 confirmed at the interval confirming step, and an embolization substance-containing liquid injection amount decision step of deciding the injection amount of the cyanoacrylate-based embolization substance-containing liquid to be injected at the embolization substance injection step.

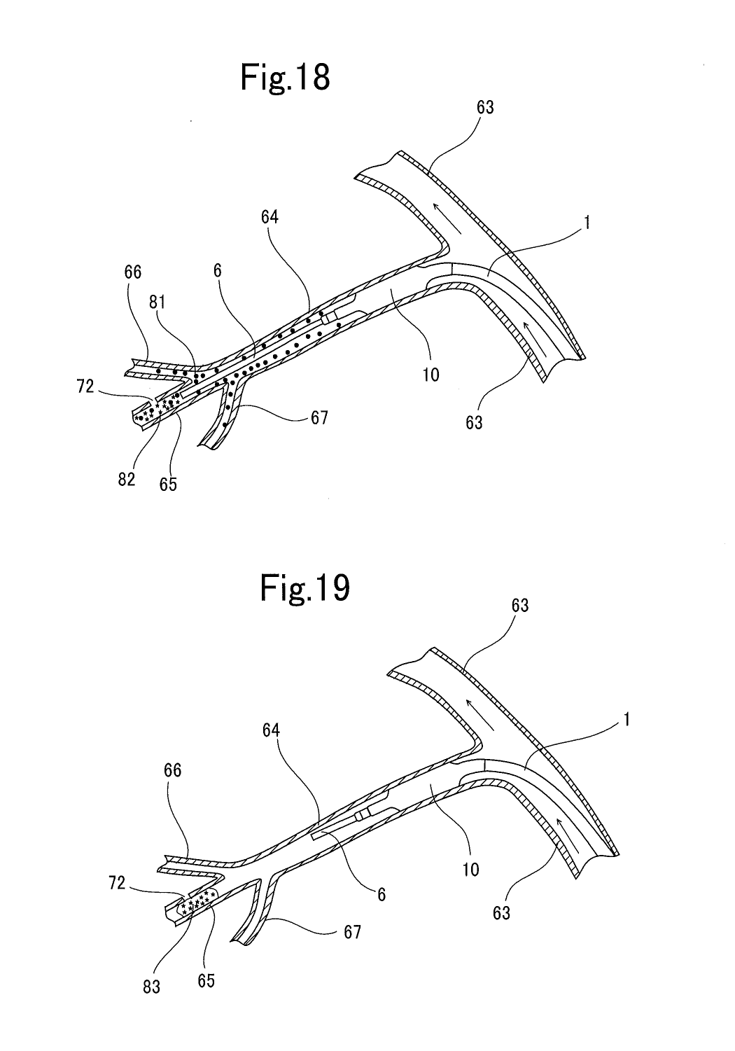

Thereafter a tubular catheter insertion step is performed. As shown in FIG. 18, at the tubular catheter insertion step, the tubular catheter 6 is inserted into the balloon catheter 1, the distal portion of the tubular catheter 6 is projected from the distal end of the balloon catheter 1 to insert the distal portion thereof into the branching blood vessel 65 and locate the distal portion thereof at a portion which is disposed in the neighborhood of the lesion site 72 and a little proximally from the lesion site 72. Although it is preferable to perform the tubular catheter insertion step after the glucose solution injection step finishes, the tubular catheter insertion step may be performed before the glucose solution injection step is performed. It is also possible to perform a glucose solution filling step of filling the glucose solution into the tubular catheter 6 before inserting the tubular catheter 6 into the balloon catheter 1. It is preferable that the lumen of the tubular catheter 6 is filled with a glucose solution liquid beforehand. The filled glucose solution liquid in the tubular catheter 6 is pushed out by injecting cyanoacrylate-based embolization substance-containing liquid at the following embolization substance injection step.

Thereafter the embolization substance injection step of discharging the cyanoacrylate-based embolization substance-containing liquid from the distal end of the tubular catheter is performed. At the embolization substance injection step, the cyanoacrylate-based embolization substance-containing liquid is discharged from the distal end of the tubular catheter 6 by using the lumen of the tubular catheter 6, after the glucose solution is injected into the above-described region of the branching blood vessel 65. At the embolization substance injection step, the cyanoacrylate-based embolization substance-containing liquid is injected into the branching blood vessel 65, having the lesion site 72, in which the blood diluted by the injected glucose solution is filled such that the cyanoacrylate-based embolization substance-containing liquid flows toward the lesion site 72. As shown in FIG. 18, the cyanoacrylate-based embolization substance-containing liquid 82 discharged from the distal end of the tubular catheter 6 at the embolization substance injection step is injected into the blood vessel in which a glucose solution 81 has been filled. The presence of the glucose solution in the blood restricts contact between the cyanoacrylate-based embolization substance and cations in the blood, thus delaying the hardening of the cyanoacrylate-based embolization substance. Therefore the cyanoacrylate-based embolization substance-containing liquid does not harden immediately after the cyanoacrylate-based embolization substance-containing liquid is injected into the above-described predetermined region, but can be securely injected into the predetermined region 72 to be embolized.

Thereafter the embolization substance-hardening step of hardening the embolization substance is performed. The embolization substance-hardening step is performed by keeping a blood flow shut-off state (in other words, expanded state of the balloon 10) and leaving the blood flow shut-off state for a predetermined period of time, after the discharge of the cyanoacrylate-based embolization substance-containing liquid finishes. The glucose solution diffuses gradually. Thereby the contact between the cyanoacrylate-based embolization substance and cations in the blood progresses. As a result, the cyanoacrylate-based embolization substance hardens. The branching blood vessel 65 having the lesion site 72 is embolized with the embolized site 83 formed of the hardened cyanoacrylate-based embolization substance, as shown in FIG. 19. It is possible to perform the tubular catheter moving backward step of moving the distal portion of the tubular catheter backward by pulling the tubular catheter toward the proximal side thereof immediately after the embolization substance is discharged at the embolization substance injection step or while the embolization substance-hardening step is being performed.

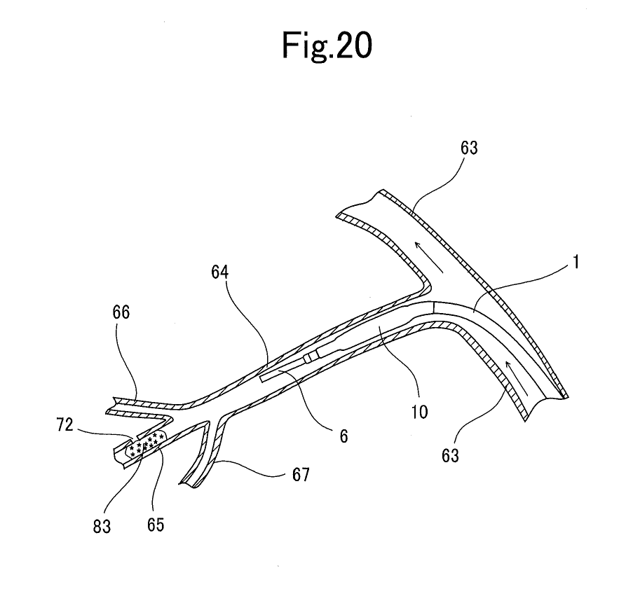

Thereafter a catheters removal step is performed. As shown in FIG. 20, at the catheters removal step, after the embolization substance-hardening step finishes, the balloon catheter 1 and the tubular catheter 6 are removed from the blood vessel after the balloon 10 contracts.

To harden the cyanoacrylate-based embolization substance more reliably, in other words, to form the perfect embolized site, after the embolization substance-hardening step is performed by keeping a state in which the balloon 10 is expanded, an auxiliary embolization substance hardening step of circulating a small amount of blood to the embolized site 83 may be performed by contracting and expanding (in other words, pumping) the balloon 10 once or plural times by keeping the state in which the balloon catheter 1 has been disposed.

In the blood vessel embolization method of the present invention, it is possible to perform the interval confirming step of confirming an approximate interval between the lesion site 72 and the distal end of the balloon catheter 1 and the glucose solution injection amount decision step of deciding the amount of the glucose solution to be injected at the glucose solution injection step in consideration of the approximate interval and the branching blood vessels 66 and 67 confirmed at the interval confirming step.

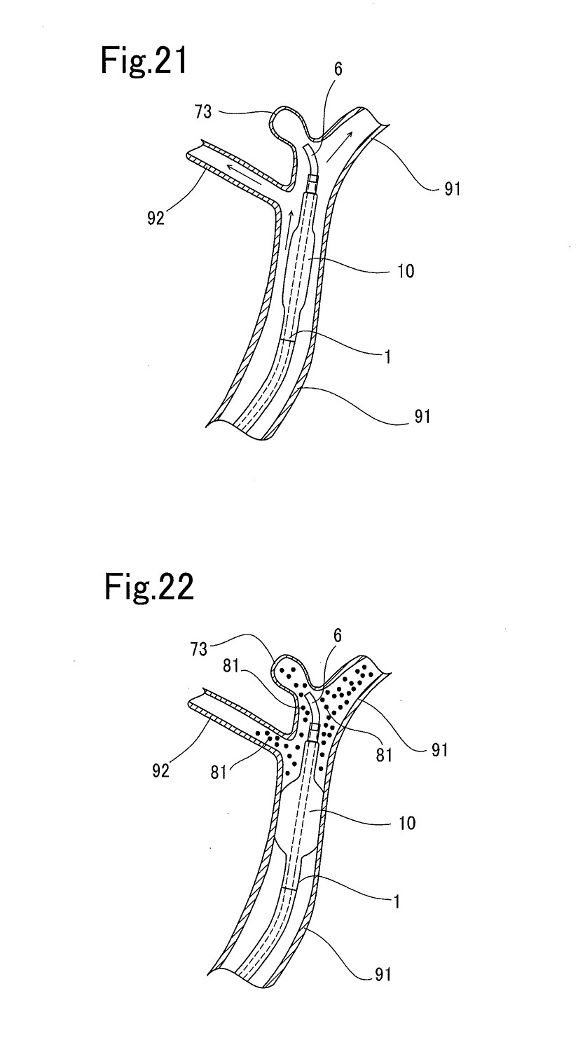

Another embodiment of the second blood vessel embolization method of the present invention is described below with reference to FIGS. 21 through 26.

In a case shown in FIG. 21, a saccular aneurysm 73 is formed in the neighborhood of the branching portion of an epigastric artery 91. The lesion site area 73 is identified by the angiographic method in which the angiographic catheter and the contrast medium are used.

Similarly to the above-described blood vessel embolization methods, in the blood vessel embolization method of this embodiment, includes the balloon catheter insertion step, the tubular catheter insertion step, the blood flow shut-off step, the glucose solution injection step, the embolization substance injection step of discharging the cyanoacrylate-based embolization substance-containing liquid from the distal end of the tubular catheter after the injection of the glucose solution finishes, the embolization substance-hardening step of hardening the embolization substance by maintaining the blood flow shut-off state of the blood vessel after the discharge of the embolization substance finishes, and the catheters removal step, including an operation of contracting the balloon, of removing the balloon catheter and the tubular catheter from the blood vessel after the embolization substance-hardening step finishes.

As shown in FIG. 21, at the balloon catheter insertion step, the balloon catheter is inserted into the blood vessel 91 having the lesion site 73 (aneurysm) in such a way that the balloon 10 is located at a portion spaced a little proximally from the branching portion close to the aneurysm 73. Thereafter at the position where the balloon catheter is disposed, the blood flow shut-off step of shutting off the blood flow by injecting the fluid into the balloon expansion lumen to expand the balloon. By performing the blood flow shut-off step, the blood flow in the blood vessel 91 and that in the branching blood vessel 92 are shut off.

Thereafter the glucose solution injection step is performed by using the main lumen 21 of the balloon catheter. The glucose solution injection step is performed so that a discharged glucose solution arrives at the lesion site 73. Also, as shown in FIG. 22, at the glucose solution injection step, the glucose solution is filled in a region from the expanded balloon 10 to a position distal from the balloon 10 to such an extent that the glucose solution is disposed distal from the lesion site 73. In this case, the glucose solution flows not only into the blood vessel 91 having the lesion site 73, but also into the branching blood vessel 92.

In the blood vessel embolization method of this embodiment, it is preferable to perform the interval confirming step of confirming the approximate interval between the lesion site 73 and the distal end of the balloon catheter 1, the glucose solution injection amount decision step of deciding the injection amount of the glucose solution to be injected at the glucose solution injection step in consideration of the approximate interval and the branching blood vessel 92 confirmed at the interval confirming step, and the step embolization substance-containing liquid injection amount decision step of deciding the injection amount of the cyanoacrylate-based embolization substance-containing liquid to be injected at the embolization substance injection step.

Thereafter the tubular catheter insertion step is performed. As shown in FIG. 22, at the tubular catheter insertion step, the tubular catheter 6 is inserted into the balloon catheter 1, the distal portion of the tubular catheter 6 is disposed in the neighborhood of the opening of the lesion site 73 with the distal portion of the tubular catheter 6 being projected from the distal end of the balloon catheter 1. Although it is preferable to perform the tubular catheter insertion step after the glucose solution injection step finishes, the tubular catheter insertion step may be performed before the glucose solution injection step or the blood flow shut-off step is performed. It is also preferable to perform a glucose solution filling step of filling the glucose solution into the tubular catheter 6 before inserting the tubular catheter 6 into the balloon catheter 1. It is preferable that the lumen of the tubular catheter 6 is filled with a glucose solution liquid beforehand. The filled glucose solution liquid in the tubular catheter 6 is pushed out by injecting cyanoacrylate-based embolization substance-containing liquid at the following embolization substance injection step.

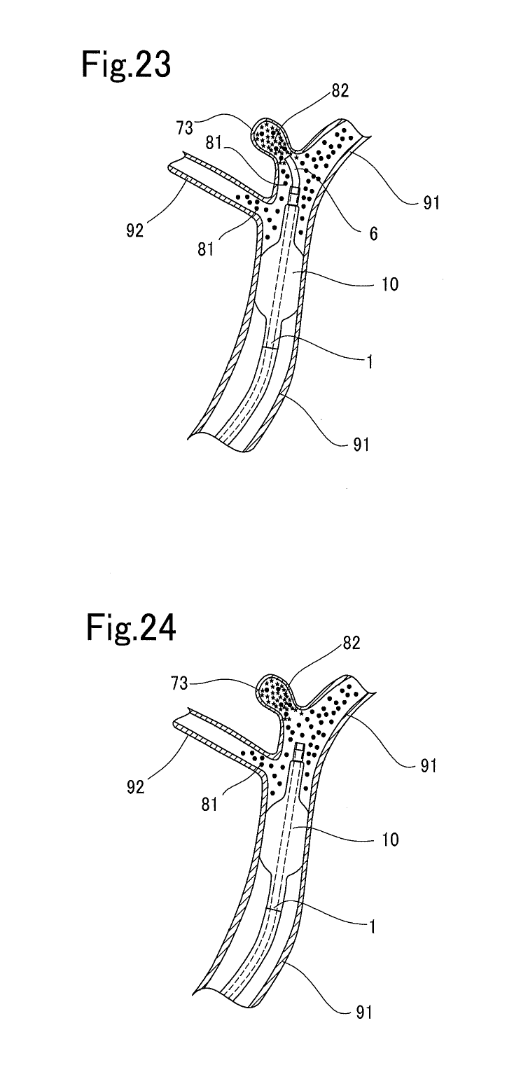

Thereafter the embolization substance injection step of discharging the cyanoacrylate-based embolization substance-containing liquid 82 from the distal end of the catheter is performed. At the embolization substance injection step, the cyanoacrylate-based embolization substance-containing liquid 82 is discharged from the distal end of the tubular catheter 6 by using the lumen (not shown in the drawings) of the tubular catheter 6 after the injection of the glucose solution finishes. At the embolization substance injection step, the cyanoacrylate-based embolization substance-containing liquid 82 is injected into the lesion site 73 in which blood diluted by the injected glucose solution is filled, as shown in FIG. 23. The presence of the glucose solution at the lesion site 73 and in the peripheral portion thereof restricts the contact between the cyanoacrylate-based embolization substance and cations in the blood, thus delaying the hardening of the cyanoacrylate-based embolization substance. Therefore the cyanoacrylate-based embolization substance-containing liquid 82 does not harden immediately after the cyanoacrylate-based embolization substance-containing liquid 82 is injected into the lesion site 73, but the cyanoacrylate-based embolization substance-containing liquid can be securely injected to the lesion site 73 to be embolized.

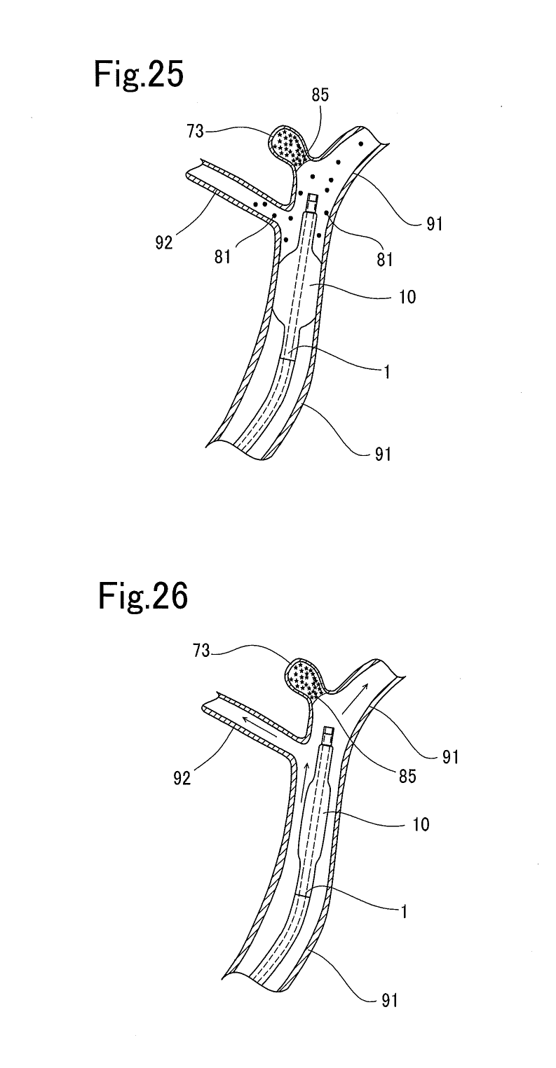

Thereafter the embolization substance-hardening step is performed. The embolization substance-hardening step is performed by keeping a blood flow shut-off state (in other words, expanded state of the balloon 10) and leaving the blood flow shut-off state for a predetermined period of time, after the discharge of the cyanoacrylate-based embolization substance-containing liquid finishes. The glucose solution diffuses gradually. Thereby the contact between the cyanoacrylate-based embolization substance and the cations in the blood progresses. As a result, the cyanoacrylate-based embolization substance hardens. As shown in FIG. 25, the lesion site 73 is embolized by the embolized site 85 formed of the hardened cyanoacrylate-based embolization substance. Unlike the above-described embolized site 83, the embolized site 85 does not shut off the blood flow in the blood vessel 91, but embolizes only the lesion site 73 (aneurysm). As shown in FIG. 24, it is possible to perform the tubular catheter moving backward step of moving the distal portion of the tubular catheter backward by pulling the tubular catheter toward the proximal side of the balloon catheter 1 immediately after the embolization substance is discharged at the embolization substance injection step or while the embolization substance-hardening step is being performed.

Thereafter a catheters removal step is performed. As shown in FIG. 26, at the catheters removal step, after the embolization substance-hardening step finishes, the balloon catheter 1 and the tubular catheter 6 are removed from the blood vessel after the balloon 10 contracts.

To harden the cyanoacrylate-based embolization substance more reliably, in other words, to form the perfect embolized site, after the embolization substance-hardening step is performed by keeping a state in which the balloon 10 is expanded, an auxiliary embolization substance hardening step of circulating a small amount of blood to the lesion site 73 may be performed by contracting and expanding (in other words, pumping) the balloon 10 once or plural times by keeping the state in which the balloon catheter 1 has been disposed.

What is claimed is:

1. A blood vessel embolization method using a balloon catheter having a main lumen and a balloon expansion lumen, comprising:
a balloon catheter insertion step of inserting said balloon catheter into a blood vessel of a human or an animal having a lesion site and disposing a balloon of said balloon catheter at a portion located at a proximal side of the blood vessel and in a neighborhood of said lesion site thereof or at a portion not spaced at a long distance from said lesion site and disposed at said proximal side of said blood vessel;
a blood flow shut-off step of shutting off a blood flow in said blood vessel by injecting a fluid into said balloon expansion lumen to expand said balloon;
a glucose solution injection step of discharging a glucose solution from a distal end of said balloon catheter by using said main lumen of said balloon catheter in a state in which said blood flow inside said blood vessel is shut off;
an embolization substance injection step of discharging a cyanoacrylate-based embolization substance-containing liquid from said distal end of said balloon catheter by using said main lumen of said balloon catheter after said glucose solution injection step;
an embolization substance-hardening step of hardening a embolization substance by maintaining a blood flow shut-off state of said blood vessel after said embolization substance injection step; and
a balloon catheter removal step of removing said balloon catheter from said blood vessel after said balloon is contracted after said embolization substance-hardening step.

2. A blood vessel embolization method using a balloon catheter, according to claim 1, which is a balloon catheter in which an outer diameter of an outer tube is not more than 1.2 mm.

3. A blood vessel embolization method using said balloon catheter according to claim 1, wherein said cyanoacrylate-based embolization substance is N-butyl-cyanoacrylate.

4. A blood vessel embolization method using said balloon catheter according to claim 1, wherein said cyanoacrylate-based embolization substance-containing liquid contains a contrast medium substance.

5. A blood vessel embolization method using said balloon catheter according to claim 1, wherein said glucose solution injection step is performed so that a discharged glucose solution arrives at said lesion site.

6. A blood vessel embolization method using said balloon catheter according to claim 1, wherein at said balloon catheter insertion step, said balloon of said balloon catheter is disposed at a portion spaced slightly proximally from said lesion site of said blood vessel; at said glucose solution injection step, said glucose solution is filled in a region between said distal end of said balloon catheter and said lesion site of said blood vessel; and at said embolization substance injection step, a cyanoacrylate-based embolization substance-containing liquid is injected into said region, between said distal end of said balloon catheter and said lesion site of said blood vessel, in which said injected glucose solution is filled such that a cyanoacrylate-based embolization substance-containing liquid flows toward said lesion site.

7. A blood vessel embolization method using said balloon catheter according to claim 1, wherein said lesion site is a bleeding site; wherein at said balloon catheter insertion step, said balloon is located at a portion of a branching blood vessel, having said bleeding site, which is proximal from said bleeding site thereof or at a portion of said branching blood vessel which is disposed in a neighborhood of said branching portion, having said bleeding site, which is disposed at a proximal side of said branching blood vessel.

8. A blood vessel embolization method according to claim 1, further comprises an interval confirming step of confirming an approximate interval between said lesion site and said distal end of said balloon catheter, and a glucose solution injection amount decision step of deciding an injection amount of said glucose solution to be injected at said glucose solution injection step in consideration of said approximate interval confirmed at said interval confirming step.

9. A blood vessel embolization method according to claim 8, further comprises an embolization substance-containing liquid injection amount decision step of deciding an injection amount of said cyanoacrylate-based embolization substance-containing liquid to be injected at said embolization substance injection step in consideration of said approximate distance confirmed at said interval confirming step.

10. A blood vessel embolization method using a balloon catheter having a main lumen and a balloon expansion lumen and a tubular catheter insertable into said balloon catheter, comprising:
a balloon catheter insertion step of inserting said balloon catheter into a blood vessel of a human or an animal having a lesion site and disposing a balloon of said balloon catheter at a portion located at a proximal side of the blood vessel and in a neighborhood of said lesion site thereof or at a portion not spaced at a long distance from said lesion site and disposed at said proximal side of said blood vessel;
a tubular catheter insertion step of inserting said tubular catheter into said balloon catheter and locate a distal portion of said tubular catheter at a position close to said lesion site;
a blood flow shut-off step of shutting off a blood flow in said blood vessel by injecting a fluid into said balloon expansion lumen to expand said balloon;
a glucose solution injection step of discharging a glucose solution from a distal end of said balloon catheter in a state in which said blood flow inside said blood vessel is shut off;
an embolization substance injection step of discharging a cyanoacrylate-based embolization substance-containing liquid from a distal end of said tubular catheter after said glucose solution injection step;
an embolization substance-hardening step of hardening a embolization substance by maintaining a blood flow shut-off state of said blood vessel after said embolization substance injection step; and
a catheters removal step, including contraction of said balloon, of removing said balloon catheter and said tubular catheter from said blood vessel after said embolization substance-hardening step.

11. A blood vessel embolization method using said balloon catheter according to claim 10, wherein a catheter moving backward step of moving said distal portion of said catheter backward is performed by pulling said catheter toward a proximal side thereof immediately after said embolization substance is injected at said embolization substance injection step or while said embolization substance-hardening step is being performed.

12. A blood vessel embolization method using said balloon catheter according to claim 10, wherein said glucose solution injection step is performed so that a discharged glucose solution arrives at said lesion site.

13. A blood vessel embolization method using said balloon catheter according to claim 10, wherein at said balloon catheter insertion step, said balloon of said balloon catheter is disposed at a portion spaced slightly proximally from said lesion site of said blood vessel; at said glucose solution injection step, said glucose solution is filled in a region between said distal end of said balloon catheter and said lesion site of said blood vessel; and at said embolization substance injection step, a cyanoacrylate-based embolization substance-containing liquid is injected into said region and toward said lesion site.

14. A blood vessel embolization method according to claim 10, further comprises an interval confirming step of confirming an approximate interval between said lesion site and said distal end of said balloon catheter, and a glucose solution injection amount decision step of deciding an injection amount of said glucose solution to be injected at said glucose solution injection step in consideration of said approximate interval confirmed at said interval confirming step.

15. A balloon catheter, for a blood vessel embolization method, having a main lumen and a balloon expansion lumen:
said blood vessel embolization method comprising: a balloon catheter insertion step of inserting said balloon catheter into a blood vessel of a human or an animal having a lesion site and disposing a balloon of said balloon catheter at a portion located at a proximal side of the blood vessel and in a neighborhood of said lesion site thereof or at a portion not spaced at a long distance from said lesion site and disposed at said proximal side of said blood vessel; a blood flow shut-off step of shutting off a blood flow in said blood vessel by injecting a fluid into said balloon expansion lumen to expand said balloon; a glucose solution injection step of discharging a glucose solution from a distal end of said balloon catheter by using said main lumen of said balloon catheter in a state in which said blood flow inside said blood vessel is shut off; an embolization substance injection step of discharging a cyanoacrylate-based embolization substance-containing liquid from said distal end of said balloon catheter by using said main lumen of said balloon catheter after said glucose solution injection step; an embolization substance-hardening step of hardening a embolization substance by maintaining a blood flow shut-off state of said blood vessel, after said embolization substance injection step; and a balloon catheter removal step of removing said balloon catheter from said blood vessel after said balloon is contracted after said embolization substance-hardening step,
said balloon catheter comprising: an inner tube having a main lumen; an outer tube provided coaxially with said inner tube and forming a balloon expansion lumen between an outer surface of said inner tube and an inner surface thereof; a balloon expandable and communicating with said balloon expansion lumen; and a hub fixed to a proximal portion of said inner tube and that of said outer tube; and
said hub comprising a first port, linearly communicating with a proximal portion of said main lumen, to which a first liquid injection tool in which a glucose solution is filled can be connected; a second port, obliquely communicating with said main lumen or an inside of said first port, to which a second liquid injection tool in which a cyanoacrylate-based embolization substance-containing liquid is filled can be connected; and a third port, communicating with said balloon expansion lumen, to which a liquid injection tool for injecting a balloon expansion fluid to said third port can be connected.

16. A balloon catheter for a blood vessel embolization method according to claim 15, wherein said outer tube has a distal end at a position backward from said distal end of said inner tube in a predetermined length; and
a distal portion of said balloon is fixed to said inner tube, and a proximal portion of said balloon is fixed to a distal portion of said outer tube.

17. A balloon catheter for a blood vessel embolization method according to claim 15, wherein said hub is so constructed that said first liquid injection tool in which said glucose solution is filled and said second liquid injection tool in which said cyanoacrylate-based embolization substance-containing liquid is filled can be simultaneously connected to said first and second ports of said hub respectively.

18. A balloon catheter for a blood vessel embolization method according to claim 15, wherein said balloon catheter in which an outer diameter of an outer tube is not more than 1.2 mm.
